(19) United States
(12) Patent Application Publication
(10) Pub. No.: US 2010/0104674 A1
(43) Pub. Date: Apr. 29, 2010

(54) COMPOSITION FOR THE TREATMENT OF HYPERTENSION

(75) Inventors: Yousef Abdullatif Jamel, Wien (AT); Rafida Mohammed Ahmed Al-Dabooni, Sharjha (AE); Karam Mashaal Hamed al Sari, Sharjha (AE)

Correspondence Address: BUCHANAN, INGERSOLL & ROONEY PC, POST OFFICE BOX 1404, ALEXANDRIA, VA, 22313-1404, US

(73) Assignee: Auron Investments Ltd., Hamilton (BM)

(21) Appl. No.: 12/325,845

(22) Filed: Dec. 1, 2008

(30) Foreign Application Priority Data
Oct. 27, 2008 (GB) 0819635.4
Nov. 14, 2008 (GB) 0820885.2

(51) Int. Cl. A61K 36/28; A61P 9/12

(52) U.S. Cl. 424/764

(57) ABSTRACT

The present invention is directed to a composition for the treatment of hypertension and/or of the symptoms associated with hypertension and/or of diseases caused by hypertension.

This composition contains an extract of at least, one Chamomilla plant and/or of at least one Achillea plant.

### FIELD OF THE INVENTION

The present invention is directed to a composition for the treatment of hypertension .and/or of the symptoms associated with hypertension .and/or of diseases, caused by hypertension.

The present invention is also directed to the use of a composition containing ah extract of at least one Chamomilla plant and/or of at least one Achillea plant for the preparation of a medicament for the treatment of hypertension and/or of the symptoms associated with hypertension and/or of diseases caused by hypertension.

Furthermore, the present invention is directed to a method for the treatment of the human or animal body suffering under hypertension and/or under the symptoms associated with hypertension and/or under diseases caused by hypertension.

### BACKGROUND OF THE INVENTION

The therapeutic properties of various plants, have been known for millennia. Even at the moment when the present application has. been filed the nature of the effective components and their properties are in many cases only little understood.

Among the many plants used for therapeutic purposes Chamomilla plants are quite common.

There exists a great variety of Chamomilla plants which belong to the family of the Asteraceae. A part of the Asteraceae family is the genus Anthemis.

Anthemis nobilis L. and Matricaria recutita L. have found widespread therapeutic applications. It is often only a specific part of the Chamomilla plants or even an extract thereof which is of interest in therapeutic treatment of a specific disorder and/or of a disease. Such extracts contain the active ingredients of a plant or of a part thereof, often a mixture of the essential oils.

Chamomilla oils can be extracted .from the flowers of the Chamomilla plants .by steam distillation. Anthemis nobilis L. yields about 1.7 v/m % of essential oil, and Matricaria recutita L. yields about 0.2-0.4 v/m % of essential oil.

The essential oil of Roman chamomile (Matricariae romanae aetheroleum) is produced from Anthemis nobilis L. (Synonym: Chamaemelum nobile (L.) All.). The essential oil of Roman chamomile has a sweet, apple-like fragrance and a very light, clear blue colour. Roman chamomile is cultivated for example in Germany, France, Spain, Italy and Morocco.

The major chemical components of Roman chamomile oil are α-pinene, camphene, β-pinene, sabinene, myrcene, 1,8-cineole, γ-terpinene, caryophyllene, propyl angelate and isobutyl angelate.

The main therapeutic properties of Roman chamomile oil are the following: antiseptic, anti-inflammatory, bactericidal, carminative, sedative, digestive and vulnerary (Hunnius, Pharmazeutisches Wörterbuch, 7. Auflage; 309; Walter de Gruyter; Berlin; New York 1993 and R. Hänsel, O. Sticher, Pharmakognosie-Phytopharmazie, 8; Auflage, 1066-1068; Springer Medizin Verlag Heidelberg 2007).

The essential oil of German chamomile (Matricariae aetheroleum) is produced from Matricaria recutita L. (Synonym,: Chamomilla recutica (L.) Rauschert). The essential oil of German chamomile has a sweet, straw-like fragrance and a dark blue colour. German chamomile is cultivated for example in Eastern Europe, in particular Hungary, France arid Egypt. The major chemical constituents of German chamomile oil are chamazulene, α-bisabolol, bisabolol oxide A, bisabolol oxide B and bisabolone oxide A.

The main therapeutic properties of German chamomile oil are the following: anti-inflammatory, bactericidal, carminative, digestive, sedative, stomachic and vulnerary (Hunnius, Pharmazeutisches Wörterbuch, 71. Auflage; 309; Walter de Gruyter; Berlin; New York: 1993 and P. Hänsel, O. Sticher, Pharmakognosie-Phytopharmazie, 8. Auflage., 1132-1133; Springer Medizin Verlag Heidelberg 2007).

Hypertension is defined as a constant increase of the blood pressure in the arterial vascular, system exceeding a systolic blood pressure of 139 mm Hg and a diastolic blood pressure of 89 mm Hg. According to clinical and organopathogenetic aspects hypertension has. to . be distinguished into the essential hypertension (accounting for 90% of all, types), the renal hypertension (6 to 8%) and the endocrine, the cardiovascular and the neurogenic hypertension (together less or equal to 3%). Hypertension triggers the pathogenesis of arteriosclerosis, coronary insufficiency and renal insufficiency (Mutschler Arzneimittelwirkungen; Lehrbuch der Pharmakologie und Toxikologie;. 8. Ausgabe, 571-574;. Stuttgart; Wiss. Veil. Ges., 2001).

It is an object of the present invention to provide a composition for the treatment of hypertension and/or of the symptoms associated, with hypertension and/or of diseases caused by hypertension.

It is an object of the present invention to provide a composition comprising the essential oil of Chamomilla, preferably the essential oil of Matricaria recutita L. (Matricariae aetheroleum) and/or of the essential oil of Anthemis nobilis L. (Matricariae romanae aetheroleum) for the treatment of hypertension and/or of the symptoms associated with hypertension and/or of diseases caused by hypertension.

It is a further object of the present invention to provide a method for the treatment of the human or animal, body suffering under hypertension and/or under the symptoms associated with hypertension and/or under diseases caused by hypertension.

It is also an object of the present, invention to provide the use of said composition for the treatment of the human or animal body suffering under hypertension and/or under the symptoms associated with hypertension and/or under diseases caused, by hypertension.

These objects are attained -with the present invention.

The invention is characterized by the characteristics as defined in the independent claims.

Preferred embodiments are defined in. the dependent claims.

### BRIEF DESCRIPTION OF THE INVENTION

It has now surprisingly been found that an extract of at least one Chamomilla plant and/or of at least one Achillea plant, especially an extract, comprising the essential oils of the Chamomilla plant and/or of the Achillea plant, obtainable for example by steam, distillation, is effective, against hypertension and/or of: the symptoms associated with hypertension and/or of diseases caused by hypertension.

This extract exhibits activity for the lowering of blood pressure, in particular pathologically increased blood pressure.

It was found quite surprisingly, that the treatment of patients suffering from hypertension with Matricariae romanae aetheroleum (Roman chamomile oil) resulted in a marked reduction of their blood pressure.

More surprisingly it was found, that a composition of Matricariae romanae aetheroleum and Matricariae aetheroleum (German chamomile oil) was even more effective in reducing elevated blood pressure levels, thus developing synergistic effects with respect of said activity.

Quite more surprisingly it was found, that the patients, could remove all antihypertensive drugs under treatment with the chamomile oil and kept the normal blood pressure. It can therefore been expected that the treatment of patients suffering from hypertension with the inventive composition will also treat symptoms and side effects of hypertension and will prevent or treat diseases caused by hypertension.

A particular advantage of the present .invention consists therein that the Chamomilla oil (chamomile extract) according to the present invention can be used on its own without any pharmaceutical aids, carriers or excipients and simply be applied to the skin of a patient in order to exert its anti-hypertensive effect and/or to treat the symptoms of hypertension.

### DETAILED DESCRIPTION OF THE INVENTION

The present invention is based on data obtained, using the essential oil of chamomile flower heads, preferably obtainable by steam distillation. Precisely the essential oil is composed of the volatile oil components of the flower heads of German chamomile (Matricaria recutita L.) and Roman chamomile (Anthemis nobilis L.).

Other suitable Chamomilla plants are Anthemis species, such as Anthemis arvensis L., Anthemis cotula L., Anthemis tinctoria L.

Other suitable plants are Ormenis multicaulis Br.-Bl. & Maire, Eriocephalus punctulatus DC. and Matricaria discoidea DC.

The essential oil or oils may be obtained by any suitable procedure known to those of ordinary skill in the art. The essential oil or oils may be obtained for example by using an aqueous or organic extraction medium or by using a water steam distillation.

The essential oil or oils may be separated from other components by means of filtration, chromatography, supercritical fluid extraction etc.

The compositions according to the present invention can be. formulated by methods known to those skilled in the art. Pharmaceutically acceptable auxiliary agents can be used.

The compositions according to the present invention are preferably in a form, which is suitable for injection, for rectal administration, for inhalation, for oral application or for topical application, especially transdermal therapeutic systems, TTS, dermal patches, liniments, creams, ointments, jellies, solutions or suspensions.

The exact dosage and frequency of administration, depend on several factors. For example it was found that the transdermal administration of 1.5 ml of an inventive composition twice daily resulted in a marked reduction of the increased blood pressure in hypertensive patients.

In the following part are described possible embodiments of the present invention.

The following examples illustrate the present invention.

### EXAMPLES

### Example 1

### Dermal Patch with a Composition of 1 Essential Oil

.1.5 ml of Matricariae romanae aetheroleum (CAS No.: 3015-92-7) were placed drop by drop on the cotton fleece of a Mepore® Pro Patch (9×10 cm; Mölnycke Health Care Lot No 0802-9783) until the oil was completely absorbed.

### Example 2

### Dermal Patch with a Composition of 2 Essential Oils

.1.35 ml of Matricariae romanae aetheroleum (CAS No.: 8015-92-7) and 0.15 ml Matricariae aetheroleum (CAS No.: 8002-66-2) were carefully mixed together. The mixture was placed drop by drop on the cotton fleece of a Mepor® Pro Patch (9×10 cm; Mölnycke Health Care Lot No 0802-9783) until the oils were completely absorbed.

### Example 3

### Liniment with a Composition of 2Essential Oils

546.875 ml Olivae oleum raffinatum (PhEur) and 390.625 ml Amygdalae oleum raffinatum (PhEur) were mixed together. 56.250 ml. Matricariae romanae aetheroleum (CAS No.: 8015-92-7) and 6.250 ml Matricariae aetheroleum (CAS No.: 8002-66-2) were mixed together and subsequently added under gentle stirring to the mixture of Olivae oleum raffinatum (PhEur) and Amygdalae oleum raffinatum (PhEur.) until a clear homogeneous solution of 1000 ml was obtained.

### Example 4

### Therapeutic Example Using the Dermal Patch from Example 1

The patient was female, 68 years old, of 75 kg weight and suffered from hypertension since 3 years. The systolic blood pressure before beginning of treatment was. 153 mm Hg and the diastolic blood pressure 88 mm Hg. Medication: None. The patient received the dermal patch according to example 1 twice daily (every twelve hours.) on the upper right arm. After a treatment period of 12 days the systolic blood pressure was 148 mm Hg and the diastolic blood pressure. 80 mm Hg.

### Example 5

### Therapeutic Example Using the Dermal Patch from Example 2

The patient was male, 51 years old, of 86 kg weight and suffered from hypertension since 5 years. The systolic blood pressure before beginning of treatment was 142 mm Hg and the diastolic blood, pressure 101 mm Hg. Medication: Covercell®. The patient received the dermal patch according to example 2 twice daily (every twelve hours) on the upper right arm. After a treatment period of 7 days the systolic blood pressure was 136 mm Hg and the diastolic blood pressure 95 mm Hg. At this time point the Covercell® medication was stopped but the treatment with the dermal patch continued. After a treatment period of 10 further days the systolic blood pressure was 124 mm Hg and the diastolic blood pressure 91 mm Hg.

### Example 6

### Therapeutic Example Using the Dermal Patch from Example 2

The patient was female, 71 years old, of 75 kg weight and suffered from hypertension since 10 years. The systolic blood pressure before beginning of treatment was 170 mm Hg and the diastolic blood pressure 70 mm Hg. Medication: Brindine®. The patient received the dermal patch according to example 2 twice daily (every twelve hours) on the upper right arm. After a treatment period of 9 days the systolic blood pressure was 147 mm Hg and the diastolic blood pressure 65 mm Hg.

### Example 7

### Therapeutic Example Using the Liniment from Example 3

The patient was female, 51 years old, of 70 kg weight and suffered from hypertension since 5 years. The systolic blood pressure before beginning of treatment was 148 mm Hg and the diastolic blood pressure 102 mm Hg. Medication: Atenolol® and Lasix®. The patient received the liniment according to example 3 in an amount of 24 ml twice dally (every twelve hours) on the back and neck. After a treatment period of 7 days the systolic blood pressure was 141 mm Hg and the diastolic blood pressure 98 mm Hg. At this time point the Atenolol® medication was stopped but the treatment with Lasix® and the liniment continued. After a treatment period of 7 further days the systolic blood pressure was 136 mm Hg and the diastolic blood pressure 93 mm Hg. At this time point the Lasix® medication was stopped but the treatment with the liniment continued. After a treatment period of 21 further days the systolic blood pressure was 121 mm Hg and the diastolic blood pressure 80 mm Hg.

### CONCLUSIONS

In the examples 1 to 3 is shown by which combinations compositions may be prepared or applications can be produced in order to meet the objects of the present invention.

The examples 4 to 7 demonstrate the antihypertensive effects of the compositions used and applications prepared according to the present invention. Comparison of example 4 with the examples 5 and 6 indicates the synergistic effect of the combination, of German camomile oil (Matricariae aetheroleum) and Roman camomile oil (Matricariae romanae aetheroleum) in reducing elevated blood pressure levels. The examples 4 to 7 show the high therapeutic potential of the compositions of the present invention and their ability to reduce high blood pressure and even to prevent an increase in blood pressure after removal of standard medications.

1. A composition, for the treatment of hypertension and/or of the symptoms associated with hypertension and/or of diseases caused by hypertension, characterised in that it contains an extract of at least one Chamomilla plant and/or of at least one Achillea plant.

2. The composition according to claim 1, characterized in that the extract comprises the oils, especially the essential oils., of the Chamomilla plant and/or of the Achillea plant.

3. The composition according to one of claims 1 to 2, characterised in that the extract is obtainable by steam distillation.

4. The composition according to one of claims 1 to 3, characterized in that the extract is obtainable from the flower heads of the Chamomilla plant and/or of the herbs and/or the flower heads of the Achillea plant.

5. The composition according to One of claims 1 to 4, characterized in that, the Chamomilla plant is selected from the group consisting of
Matricaria species, especially
Matricaria recutita L.
Matricaria discoidea DC.
Anthemis species, especially
Anthemis nobilis L.
Anthemis arvensis L.
Anthemis cotula L.
Anthemis tinctoria L.
Ormenis multicaulis Br.-Bl. & Maire
Eriocephalus punctulatus DC.,
whereby Matricaria recutita L. and Anthemis nobilis L. are especially preferred.

6. The composition according to one of claims 1 to 5, characterized in that it contains at least, one additional component selected from the group consisting of
angiotensin converting enzyme inhibitors, abbreviated as ACE inhibitors,
calcium antagonists, and
3-hydroxy-3-methylglutaryl-coenzyme-A-reductase inhibitors, abbreviated as HMG-CoA-reductase inhibitors.

7. The composition according to one of claims 1 to 6, characterized in that it contains at, least one pharmaceutical and/or dermatological acceptable auxiliary agent.

8. The composition according to one of claims 1 to 7, characterized in that it is in such a form which is suitable for injection, for rectal administration, for inhalation, for oral application or for topical application, especially transdermal therapeutic systems, TTS, dermal patches, liniments, creams, ointments, jellies, solutions or suspensions.

9. The composition according to one of claims 1 to 8, characterized in that the symptoms associated with hypertension and/or of diseases caused, by hypertension are selected from the group consisting of cardiovascular diseases, especially endothelial dysfunction, arteriosclerosis, angina pectoris, coronary heart disease and myocardial infarction, chronic headache, dizziness or vertigo, blurry or double vision, drowsiness, nausea, shortness of breath, heart palpitations, prolonged fatigue, a flushed face, nosebleeds, a strong need to urinate often, especially during the night, tinnitus.

10. The composition according to one of claims 1 to 9, characterized in that the treatment comprises the step of applying or administering the composition to the surface of the skin, preferably on an arm, the neck or on the back between the shoulders.

11. Use of a composition containing an extract of at least one Chamomilla plant and/or of at least one Achillea plant for the treatment of hypertension and/or of the symptoms associated with hypertension and/or of diseases caused by hypertension.

12. Use according to claim 11, characterized in that the composition is such a composition as defined in one of claims 2 to 10.

13. Use of a composition containing an extract of at least one Chamomilla plant and/or of at least one Achillea plant for the preparation of a medicament for the treatment of hypertension and/or of the symptoms, associated with hypertension and/or of diseases caused by hypertension.

14. Use according to claim 13, characterized in that the composition is such a composition as defined in one of claims 2 to 10.

15. A method for the treatment of the human or animal body suffering under hypertension and/or under the symptoms associated with hypertension and/or under diseases caused by hypertension, characterized in that to the patient is applied or administered a composition containing an extract; or at least one Chamomilla plant and/or of at least one Achillea plant.

16. The method according to claim 15, characterized in that the composition is such a composition as defined in one of claims 2 to 10.
